(19) United States
(12) Patent Application Publication
(10) Pub. No.: US 2011/0159556 A1
(43) Pub. Date: Jun. 30, 2011

(54) Use of hydroxyalkanoic acids as substrates for production of poly-hydroxyalkanoates by methane-oxidizing bacteria

(76) Inventors: Allison J. Pieja, Menlo Park, CA (US); Eric R. Sundstrom, Menlo Park, CA (US); Perry L. McCarty, Stanford, CA (US); Craig S. Criddle, Redwood City, CA (US)

(21) Appl. No.: 12/928,323

(22) Filed: Dec. 8, 2010

Related U.S. Application Data
(60) Provisional application No. 61/283,818, filed on Dec. 8, 2009.
(60) Provisional application No. 61/283,784, filed on Dec. 8, 2009.

(51) Int. Cl. C12P 7/62

(52) U.S. Cl. 435/135

(57) ABSTRACT

A method of biosynthesis of polyhydroxyalkanoates (PHA) is provided that includes providing a type II methanotrophic bacteria, and disposing the type II methanotrophic bacteria in an unbalanced growth condition, where the unbalanced growth condition includes a nutrient-deficient media and a hydroxyalkanoic acid, and where the nutrient-deficient media has an absence of an essential nutrient required for cell replication of the type II methanotrophic bacteria.

### CROSS-REFERENCE TO RELATED APPLICATIONS

This application claims priority from U.S. Provisional Patent Application 61/283,818 filed Dec. 8, 2009, which is incorporated herein by reference.

This application claims priority from U.S. Provisional Patent Application 61/283,784 filed Dec. 8, 2009, which is incorporated herein by reference.

### FIELD OF THE INVENTION

This invention relates generally to methods for microbial biosynthesis of biopolymers. More specifically, it relates to improved biosynthesis of polyhydroxyalkanoates (PHA).

### BACKGROUND OF THE INVENTION

As environmental concerns increase over the production and disposal of conventional petrochemical-based plastics, there is a growing incentive to find a simple method of producing inexpensive alternatives.

Bioplastics have numerous advantages over petrochemical-based plastics. Unlike petrochemical-based plastics, bioplastics rapidly biodegrade and are non-toxic. Bioplastics are derived from renewable resources, decreasing demand for non-renewable petrochemical resources. Bioplastics have lower energy inputs than petrochemical-based plastics, and their production results in lower CO₂ emissions than petrochemical plastic production. It is therefore of great interest to find improved methods for producing bioplastics.

Bioplastics may be produced using various biopolymers such as polyhydroxyalkanoates (PHA), and particularly the polymer of hydroxybutyrate, polyhydroxybutyrate (PHB). PHAs are polyesters with repeating subunits (100-30,000) that have the formula

—[O—CH(R)(CH₂)ₓCO]—.

The most common type of PHA is PHB, where R═CH₃ and x=1. Another is polyhydroxy valerate (PHV), where R═CH₂CH₃ and x=1.

The most common known methods of PHA production use pure cultures, relatively expensive fermentable substrates, such as sugar from corn, and aseptic operation. The price of PHA produced using this feedstock and methodology currently exceeds the price needed to be competitive with petrochemical-based plastics. Thus, an important challenge is to provide improved methods for producing PHAs that are more efficient and less expensive, so that bioplastics can become commercially competitive with petrochemical-based plastics.

Some methanotrophs have been shown to produce PHBs from methane under nutrient limited conditions. The PHB-producing potential of most methanotrophic species, however, remains largely unexplored, as are methods for efficient and inexpensive biosynthesis of PHB.

Petrochemical plastics do not degrade and accumulate in landfills. Even when they are recycled, they are usually downcycled. Petrochemical plastics are also produced from petroleum, which is a non-renewable, environmentally unfriendly substrate. PHA are biobased, biodegradable plastics that will not accumulate in landfills and can either be degraded to carbon dioxide and methane or broken down into their monomer units. There is a growing market for biodegradable plastics, such as PHA. The properties of PHA can be widely varied by adjusting the copolymer content, which make them ideal for various plastic applications, ranging from bottles to foams and films.

Polyhydroxyalkanoates (PHA) are microbially produced polyesters that can be harvested for use as biodegradable plastics. Type II methanotrophs are a group of methane-consuming bacteria that produce poly-hydroxybutyrate (PHB) under unbalanced growth conditions, i.e., when there is sufficient methane to meet cell requirements for energy and carbon but another nutrient necessary for cell replication is missing. Under such conditions, various metabolites of methane are biochemically converted into hydroxybutyrate. These hydroxybutyrate monomers are then incorporated into a PHB polymer.

### SUMMARY OF THE INVENTION

To address the needs in the art, a method of biosynthesis of polyhydroxyalkanoates (PHA) is provided that includes providing a type II methanotrophic bacteria, and disposing the type II methanotrophic bacteria in an unbalanced growth condition, where the unbalanced growth condition includes a nutrient-deficient media and a hydroxyalkanoic acid, and where the nutrient-deficient media has an absence of an essential nutrient required for cell replication of the type II methanotrophic bacteria.

According to one aspect of the invention, the type II methanotrophic bacteria includes pure cultures or mixed cultures of the type II methanotrophic bacteria.

In another aspect of the invention, the essential nutrient can be nitrogen, phosphorus, sulfur, iron, sodium, potassium, magnesium, copper, calcium, or manganese.

According to a further aspect of the invention, the hydroxyalkanoic acid includes but is not limited to 3-hydroxybutryate (3-HB), 3-hydroxyvalerate (3-HV), or 3-hydroxyhexanoate (3-HHx).

In another aspect of the invention, the polyhydroxyalkanoates include but are not limited to 4-hydroxybutryate (4-HB), 4-hydroxyvalerate (4-HV), or 3-hydroxyoctanoate (3-HO).

According to another aspect of the invention, the hydroxyalkanoic acid is provided with biogas. In one aspect the biogas is provided from biodegradation of organic waste.

In yet another aspect of the invention, the hydroxyalkanoic acid is provided with biogas and oxygen, or the biogas and air.

According to one aspect of the invention, the essential nutrient is provided to the type II methanotrophic bacteria in intermittent pulses.

In another aspect of the invention, a bioreactor is used for the biosynthesis of the PHA. In one aspect, the bioreactor is operated in cycles including n and n+1 cycles, where each cycle includes two periods, where in a first period of cycle n, methane and/or hydroxyalkanoic acid (s) is (are) provided in excess to the methanotrophic bacteria in the bioreactor, where no nutrients for the methanotrophic bacteria is provided, and the methanotrophic bacteria are able to accumulate polyhydroxyalkanoate (PHA) and increase in size, where in a second period nutrients are provided to the size-increased methanotrophic bacteria, where no biogas is provided to the size-increased methanotrophic bacteria, and where the first period and the second period are repeated for n+1 cycles, and where repeated cycling through the periods select for bacteria that produce enough the PHA in the first period to replicate during the second period of carbon starvation. In a further aspect, additional species of the methanotrophic bacteria are periodically introduced at a beginning of the first period of the cycle, where organisms able to produce more PHA more quickly become dominant. In one aspect, the bioreactor is operated in a sterile or non-sterile manner. In a further aspect, a portion of the size-increased methanotrophic bacteria are harvested as waste cells, where the PHB is extracted.

According to one aspect of the invention, a carbon source is supplied continuously to the type II methanotrophic bacteria, where the essential nutrient is provided to the type II methanotrophic bacteria in intermittent pulses.

According to one embodiment, the invention further includes providing acrylic acid that is disposed to inhibit beta-oxidation, where the acrylic acid can include prop-2-enoic acid.

In yet another aspect of the invention, the essential nutrient is provided to the type II methanotrophic bacteria in intermittent pulses, where either carbon or oxygen are disposed for limiting the growth conditions during the period of nutrient sufficiency, and the bacteria is subjected to alternating periods of carbon or oxygen limitation and nutrient limitation.

### BRIEF DESCRIPTION OF THE DRAWINGS

FIG. 1 shows is a schematic diagram of two cycles in a sequence of bioreactor cycles in which each cycle includes a first period of carbon surplus and PHA production and a second period of carbon starvation and cell division, according to one embodiment of the current invention.

FIG. 2 shows a schematic diagram of a sequencing batch reactor for PHB production from methane, according to an embodiment of the invention.

FIG. 3 shows a flow diagram of a carbon cycle of the step of transforming methane into PHB, according to one embodiment of the current invention.

FIG. 4 shows a schematic drawing of PHB production through continuous methane addition with intermittent N addition, according to one embodiment of the current invention.

### DETAILED DESCRIPTION

The current invention is a method of biosynthesis of polyhydroxyalkanoates (PHA) that includes providing one or more species of type II methanotrophic bacteria, and disposing the type II methanotrophic bacteria in an unbalanced growth condition, where the unbalanced growth condition includes a nutrient-deficient media and a hydroxyalkanoic acid, and where the nutrient-deficient media has an absence of an essential nutrient required for cell replication of the type II methanotrophic bacteria. In one embodiment, the feedstock is hydroxyalkanoic acids alone or in combination with methane.

In one aspect of the invention, the polyhydroxyalkanoates can include 4-hydroxybutryate (4-HB), 4-hydroxyvalerate (4-HV), or 3-hydroxyoctanoate)3-HO).

The invention includes the direct use of hydroxyalkanoic acids as substrates for PHA production, thus bypassing the need for methane conversion into hydroxyalkanoates. These hydroxyalkanoates can be produced via the depolymerization of waste products containing PHAs through the enzymatic action of PHA depolymerases or by chemical hydrolysis of PHAs. According to the invention, methanotrophs are capable of producing PHB in high yield from hydroxybutyric acid alone or a combination of methane and hydroxybutyric acid under unbalanced growth conditions, where the hydroxyalkanoic acid can be 3-hydroxybutryate (3-HB), 3-hydroxyvalerate (3-HV), or 3-hydroxyhexanoate (3-HHx).

According to another aspect of the invention, the hydroxyalkanoic acid is provided with biogas. In one aspect the biogas is provided from biodegradation of organic waste. Further, the hydroxyalkanoic acid can be provided with biogas and oxygen, or the biogas and air.

According to one embodiment, the invention further includes providing acrylic acid that is disposed to inhibit beta-oxidation, where the acrylic acid can include prop-2-enoic acid.

According to the invention, type II methanotrophic bacteria are grown to an exponential phase under conditions of balanced growth with methane as feedstock. The cultures are then subjected to conditions of unbalanced growth by transfer to media lacking a key nutrient and are provided with one or more hydroxyalkanoic acids, such as hydroxybutyric acid, either with or without a carbon source, for example methane. As an example, under such conditions, these bacteria use the hydroxyalkanoic acids and methane (when present) to produce PHA. In the presence of both methane and hydroxyalkanoic acids, yields of PHA (g PHA/g biomass) are higher and rates of PHA accumulation are faster than they would be if only methane were used as a substrate. By adding different hydroxyalkanoic acids during unbalanced growth, copolymers of hydroxyalkanoic acids (e.g. poly-hydroxybutyrate-co-valerate), are produced. Such polymers may have properties superior to PHB for some applications.

According to the invention, PHAs are produced by many bacteria under unbalanced growth conditions when they have access to surplus carbon but lack an essential nutrient, such as phosphorus, nitrogen, sulfur, iron, sodium, potassium, magnesium, copper, calcium, or manganese. Under these conditions, the bacteria hoard the carbon, storing it as intracellular PHA granules. The granules are consumed when supplies of carbon and energy become limiting or when the limiting nutrient or methane again become available.

The addition of hydroxyalkanoic acids can be used to produce PHA, which can in turn be used as biodegradable plastics, in methanotrophic bacteria.

Embodiments of the invention directly use hydroxyalkanoic acids by methanotrophs to produce PHA, and are capable of producing PHA other than PHB by methanotrophs.

Several advantages enabled by the invention, such as the addition of hydroxybutyrate in combination with methane increases the rate of PHB production and the overall yield of PHB in methanotrophic bacteria. Further, by using hydroxyalkanoic acids as substrates PHA are enabled to be recycled by merely breaking them down to their monomer units, as opposed to being completely degraded to carbon dioxide and methane. This allows for more efficient recycling of biodegradable plastics. It also eliminates the problem of downcycling that is common in many petrochemical-based plastics. Another advantage is that the addition of various hydroxyalkanoic acids in combination with methane enables the production of copolymers, which may have superior properties to PHB (e.g. they are more easily processed and they have higher ductility than pure PHB), from methanotrophs. Until now, it has not been possible to produce PHA other than PHB in methanotrophic bacteria. According to one embodiment, since methane is an inexpensive carbon source that is often considered a waste gas, it is an ideal substrate for PHB production, and utilizing hydroxyalkanoic acids in conjunction with methane allows for the production of more valuable products in the form of copolymers.

An example is provided that shows a mixed culture, designated as WWHS-2, produces polyhydroxybutyrate (PHB) when provided with hydroxybutyrate in the absence of nitrogen. Here, WWHS-2 was dominated by methanotrophic bacteria of the genus Methylocystis and had been previously characterized by clone libraries of the 16s rRNA and pmoA genes.

Triplicate exponential-phase batch cultures were incubated aerobically in nutrient media without nitrogen or methane and with 1 g/L hydroxybutyric acid sodium salt (HB). Under these conditions, WWHS-2 produced 14% PHB (mg PHB/mg biomass). Another set of triplicate cultures were incubated similarly, but with the addition of methane to the headspace. These cultures produced an average of 44.6% PHB. Cultures that were incubated with methane but without HB produced an average of 35.6% PHB. Thus, mixed cultures of methanotrophs can utilize HB to produce PHB in the absence of methane. They can also produce more PHB in the presence of methane and HB than in the presence of methane alone.

Another example is provided to show that a pure methanotrophic culture, Methylosinus trichosporium OB3b, can produce PHB using HB as a substrate. Here, exponential-phase batch cultures were incubated aerobically with no nitrogen and with methane and 1 g/L HB. Thus, these cultures produced an average of 10% more PHB (50% as compared to 40%) than similar cultures incubated without HB.

According to the current invention, the term “biodegradation” is defined as a breaking down of organic substances by living organisms, e.g., bacteria. In the present context, biodegradation is understood to include anaerobic fermentation. Similarly, “biosynthesis” is defined as a production of chemical compounds from simpler reagents by living organisms, e.g., bacteria.

To detail the conditions required for PHA production, the terms “growth”, “balanced growth”, and “unbalanced growth” are defined. “Growth” is defined as an increase in cell mass. This may occur through cell division (replication) and the formation of new cells during “balanced growth”, or, during “unbalanced growth”, when cellular mass increases due to the accumulation of a polymer, such as PHA. In the latter case, growth may be manifest as an increase in cell size due to the accumulation of biopolymer within the cell.

According to the invention, during balanced cell growth, all of the feedstocks (electron donors and electron acceptors) and all of the nutrients are present in the ratios required to make all of the macromolecular components of the cell. No feedstock or nutrient limits the synthesis of proteins, complex carbohydrate polymers, fats, or nucleic acids.

During unbalanced cell growth, a feedstock or nutrient needed to make one or more of the macromolecules is not present in the ratio required for balanced growth. This feedstock or nutrient therefore becomes limiting and is termed the “limiting nutrient”. Some cells may still achieve net growth under these conditions, but the growth is unbalanced, with accumulation of polymers that can be synthesized in the absence of the limiting feedstock or nutrient. These polymers include intracellular storage products, such as the polydroxyalkanoates (PHAs)—polyhydroxybutyrate (PHB), polyhdroxyvalerate (PHV), and polyhydroxyhexanoate (PHHx)—glycogen, or secreted materials, such as extracellular polysaccharide.

As an example of balanced and unbalanced growth conditions consider the nitrogen requirement for balanced cell growth. Nitrogen constitutes about 12% of dry cell weight. This means that in order to grow 100 mg/L cell dry weight, 12 mg/L of N must be supplied along with a feedstock and other nutrients in the required stoichiometric ratios. If other feedstock and nutrients are available in the quantities needed to produce 100 mg/L of cell dry weight, but less than 12 mg/L of N is provided, then unbalanced cell growth may occur, with accumulation of polymers that do not contain N. If N is subsequently provided, the stored polymer may serve as feedstock for the cell, allowing balanced growth, with replication and production of new cells.

In one aspect, the present invention provides a cost-effective method for the production of PHB using methane as a source of carbon. The methane is preferably derived from biodegradation of organic waste.

According to one aspect of the invention, a carbon source is supplied continuously to the type II methanotrophic bacteria, where the essential nutrient is provided to the type II methanotrophic bacteria in intermittent pulses. For example, the use of methane and/or volatile fatty acids as a carbon source in the feedstock makes the biosynthesis process less expensive as compared with other microbial biosynthesis processes that use more expensive carbon sources. The carbon source, such as methane, also can be continuously generated and delivered to a batch culture as a uniform feedstock for growth of methanotrophs and PHA production. The feedstock is used in aerobic microbial biosynthesis of PHA polymers using a mixed bacterial community, preferably including methanotrophs. The PHA is grown under unbalanced growth conditions, i.e., when an essential nutrient is deficient or when toxic stressors are present. The biosynthesis may be performed using a small-scale fermentation facility.

In one aspect of the invention, the essential nutrient is provided to the type II methanotrophic bacteria in intermittent pulses, where either carbon or oxygen are disposed for limiting the growth conditions during the period of nutrient sufficiency, and the bacteria is subjected to alternating periods of carbon or oxygen limitation and nutrient limitation.

Mechanical properties of a PHA resin matrix can be altered through copolymerization with other hydroxylalkanoate monomers or with reactive polymer blending. For example, when PHB is copolymerized with hydroxylvalerate (HV) or hydroxyhexanoate (HHx), the ductility, toughness, and ease of molding increase while the crystallinity and melting point decrease.

The bacterial storage polymer poly-b-hydroxybutyrate (PHB) can be extracted and used as a biodegradable plastic for applications ranging from disposable eating utensils to furniture. Commercially, PHB granules have value as plastics or resins, with properties similar to petrochemical plastics.

Turning now to a description of techniques related to the method for biosynthesis of PHA, according to the current invention.

According to some embodiments, the biosynthesis method uses a bacterial community including a variety of methanotrophs that produce the highest levels of PHB (i.e., high ratios of grams PHB to grams biomass). This can specifically include the “Type II” methanotrophs, which use a carbon assimilation pathway that feeds into the biosynthetic pathway for PHB production. Other bacteria used in the biosynthesis of PHA are enriched by growth upon the specific biodegradation products of the biodegradation process. The use of mixed bacterial cultures makes the process less expensive as compared with processes that use pure cultures by eliminating the need for maintenance of special cultures. In the current invention, the term “mixed cultures” is defined to include bacterial communities containing a variety of distinct cultures or species, irrespective of whether or not the species are well defined. The term “mixed cultures” also includes enrichment communities. These are communities of organisms subjected to selective pressures favorable for the growth of organisms that positively affect PHA production and unfavorable for the growth of organisms that negatively affect PHA production.

According to one aspect of the current invention, the bacterial cultures may be derived from biomass from various sources. Methanotrophs are found in environments where both oxygen and methane are present, often at the interface between aerobic and anaerobic zones. They are common in rice paddies, swamps and marshes, surface sediments in ponds and lakes, activated sludge, and meadow and deciduous forest soils, including freshwater, brackish, and saline environments, deserts, landfills, coal mine surfaces, and oceans. Preferable sources include those environments subject to periodic stress, such as carbon, nutrient, or oxygen limitation. Environments with periodic stresses, such as intermittent availability of methane or water, select for methanotrophs that can store carbon for use during such times of stress. It is also the case that methanotrophs isolated from environments with these different selection pressures have different rates and yields of PHB production.

Samples of methanotrophs from diverse environments are then screened for their capacity to produce PHBs and to identify cultures capable of producing commercially significant levels of PHB.

In another aspect of the invention, cultures are grown to high density, subjected to nutrient limitation (e.g., nitrogen and phosphorus), and screened for PHA production in aerobic shake flask cultures.

Methanotrophs are classified into three groups based on their carbon assimilation pathways and internal membrane structure: Type I (gamma proteobacteria), Type II (alpha proteobacteria), and a subset of type I known as Type X (gamma proteobacteria). Type I methanotrophs use the RuMP pathway for carbon assimilation whereas type II methanotrophs use the serine pathway. Type X methanotrophs use the RuMP pathway but also express low levels of enzymes found in the serine pathway. Type II methanotrophs accumulate PHB.

According to one embodiment of the invention, the essential nutrient is provided to the type II methanotrophic bacteria in intermittent pulses. In a further embodiment, methanotroph enrichments from different environments are introduced into a sequencing bioreactor with minimal media and forced to cycle between two phases: a first phase in which methane is supplied in excess while nitrogen is absent (or significantly reduced) and a second phase in which the flow of methane is stopped (or significantly reduced) and a pulse of nitrogen is added. This cycling is used to select for bacteria that store PHB when nitrogen is absent and subsequently use the PHB to produce new biomass when nitrogen is introduced to the system, thus conferring a competitive advantage on those organisms that produce higher quantities of PHB during the period of methane addition. In one embodiment, nitrogen is selected as the limiting nutrient because its absence is known to induce PHB production and it can be easily monitored. Because the reactor is intrinsically designed to select for PHB-producing methanotrophs, it can be maintained as an open, nonsterile system, thus avoiding the costs and difficulties associated with maintaining a sterile culture during industrial production of PHB. In one embodiment, shifts in community composition are monitored using a wide range of methods including terminal restriction fragment length polymorphism (T-RFLP) analysis of pmoA, clone libraries, and microarrays. System performance may be monitored by measuring the PHB content of the cells.

According to another embodiment, a methane-fed culture grown to high cell density is used to produce high percentages of PHA when supplemented with acetate and/or propionate, and limited for nitrogen or phosphorus. The most effective culture is one with high PHA yield, high rate of PHA production, high growth rate, and high fitness, allowing robust non-sterile operation. This may be achieved by allowing communities to adapt to an environment that provides a selective advantage for PHA production. The biosynthesis may be performed in a bioreactor with conditions maintained to favor high levels of PHA production under non-sterile growth conditions in rapid, high cell density fermentations.

In another aspect of the invention, a bioreactor is used for the biosynthesis of the PHA. In one aspect, the bioreactor is operated in cycles including n and n+1 cycles, where each cycle includes two periods, where in a first period of cycle n, methane is provided in excess to the methanotrophic bacteria in the bioreactor, where no nutrients for the methanotrophic bacteria is provided, and the methanotrophic bacteria are able to accumulate polyhydroxybutyrate (PHB) and increase in size, where in a second period nutrients are provided to the size-increased methanotrophic bacteria, where no biogas is provided to the size-increased methanotrophic bacteria, and where the first period and the second period are repeated for n+1 cycles, and where repeated cycling through the periods select for bacteria that produce enough the PHB in the first period to replicate during the second period of carbon starvation. In a further aspect, additional species of the methanotrophic bacteria are periodically introduced at a beginning of the first period of the cycle, where organisms able to produce more PHBs more quickly become dominant. In one aspect, the bioreactor is operated in a sterile or non-sterile manner. In a further aspect, a portion of the size-increased methanotrophic bacteria are harvested as waste cells, where the PHB is extracted.

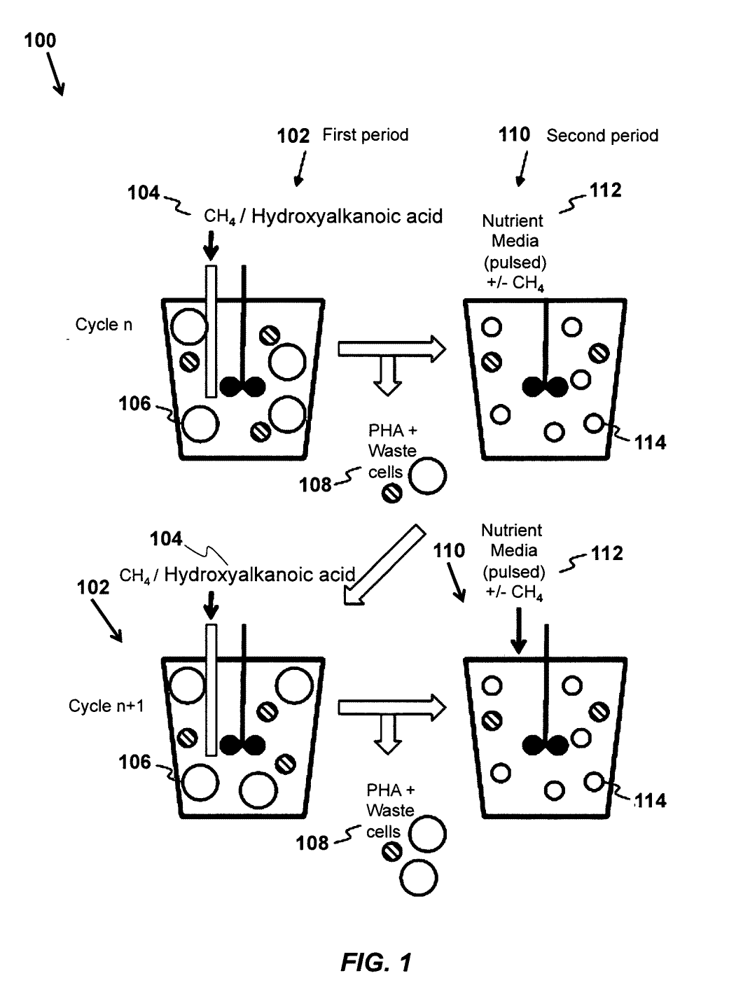

According to other embodiments of the invention, a range of bioreactor configurations may be used, including sequencing membrane bioreactors and a continuous multistage dispersed growth configuration. Preferably, the bioreactor is operated to select for bacteria that efficiently produce PHB from methane and hydroxyalkanoic acid, i.e., the bioreactor conditions select against bacteria that either do not produce PHBs from methane and hydroxyalkanoic acid, or produce them inefficiently. For example, as shown in FIG. 1, sequencing batch reactors 100 can be operated by repeatedly cycling through two periods. Cycles n and n+1, each containing two periods, are shown. In the first period 102 of cycle n, methane and/or hydroxyalkanoic acid 104 are provided in excess, but no nutrients. Methanotrophic bacteria 106 that are able to accumulate PHA under these conditions enlarge. At the end of the first period a portion of the bacteria are harvested as waste cells 108 and PHA is extracted. In the second period 110 nutrients 112 are provided with or without methane or hydroxyalkanoic acid. The methanotrophic bacteria 106 are able to use their stored PHA to replicate during this phase and to maintain cell function, while other bacteria 114 with smaller amounts of stored PHB will replicate less and are subject to cell decay as they cannot meet the energy demands for cell maintenance. The two periods are then repeated in cycle n+1, and so on. Repeated cycling through these periods will select for bacteria that produce enough PHA in the first period to replicate during the second period 110 of carbon starvation. Additional species may be periodically introduced, e.g. at the beginning of the first period 102 of a cycle. Organisms able to produce more PHA more quickly become dominant. Operating the system in a non-sterile manner ensures that the dominant species has a high relative fitness. Different methanotrophs will produce PHA with differing molecular weight distributions or different PHA polymers. Consequently, the suitability of the PHA polymers for particular target applications serves as an additional criterion for subsequent selection of cultures.

Because the rate of cellular PHB utilization for growth is directly proportional to the PHB content of a cell, cells with a higher percent of dry weight as PHB will reproduce more quickly and species that accumulate a higher percentage of PHBs will have a selective advantage over other species. This advantage can be accentuated by gradually lengthening the time period without methane or hydroxyalkanoic acid, creating a penalty for rapid PHB degradation and an incentive for PHB accumulation. In activated sludge systems, bacteria respond to periods of substrate excess (“feast”) and deficiency (“famine”) by storing PHBs during the substrate excess period and using them to make new cells during the substrate deficient period. The term “excess” in this context means that the feedstock and all other nutrients (except a limiting nutrient) are present at a level sufficient for balanced growth. The term “limited” or “deficiency” in this context means that a nutrient is present at a level that is less than needed for balanced growth. During a feedstock limitation, sufficient nutrients are present when there is enough to deplete the polymer previously stored under unbalanced growth conditions. The exact amount will depend on the amount of polymer storage that has occurred.

In addition to creating an environment that selects for methanotrophic species that produce PHBs, evolution of dominant species occur as mutations confer selective advantages on daughter strains that outcompete the parent strains. Operation evolves a robust, PHB-producing methanotroph or a mixed culture that is better able to produce PHBs than the parent culture. Species compete against one another in an environment designed to select for the desired characteristics.

As shown in FIG. 1, a set of sequencing batch reactors may be operated to select for organisms that accumulate PHBs rapidly and at high yield and to enable competition of different species of PHB-producing methanotrophic bacteria. Operation may be managed so that PHB-producing bacteria have a selective advantage over those that do not. This may be accomplished by sequencing through two periods; a first period in which methane and hydroxyalkanoic acid is present in excess but nutrients are absent and a second period in which nutrients are present but methane is absent. During the first period 102, PHB-producing bacteria accumulate PHBs; during the second period 110, the organisms that accumulated PHBs are able to produce protein and replicate while cells that did not store PHB are unable to replicate because they lack carbon. Repeated cycling between these phases with periodic biomass-wasting at the end of the methane feed period select for bacteria that produce enough PHBs to replicate during the period of carbon starvation.

The reactor is sequenced between periods of carbon excess with methane provided, and periods of carbon starvation with nutrients provided. Also shown is the effect of competition in successive cycles where the cells 114 are unable to accumulate significant quantities of PHB and thus are not able to replicate in the nutrient-sufficient phase.

In another embodiment, the system is inoculated with an enrichment. Additional species and mixed cultures are periodically introduced, at concentrations comparable to the concentration of the cells in the reactor. Prior to the addition of new cultures, an additional fraction of the existing cells are wasted. The PHB content of the wasted cells are then measured using a spectrofluorometric assay and the relative abundance of species is monitored by T-RFLP analysis. Organisms that are able to produce more PHBs more quickly and to a higher level become dominant. By operating the system in a non-sterile manner, the dominant species has a high relative fitness and has characteristics that would be desirable in an industrial system. Regularly obtained samples may be archived to permit detailed analyses of shifts in community structure that may correspond to enhancements or changes in PHB production.

According to the invention, PHAs from the most promising cultures are characterized for monomer composition, molecular weight distribution, and other parameters important to bioplastic applications. These results assist in the identification of cultures and strains for optimization of bioreactor operation and scale-up.

Information on phylogeny can be used to identify organisms, determine ecological relationships, and optimize PHB production.

Desired reactor configurations and operation select for the most promising culture that enables high levels of PHA production with minimal energy inputs. According to one aspect of the invention, also of interest are cultures that produce PHA polymer blends or copolymers that are particularly well suited for specific applications.

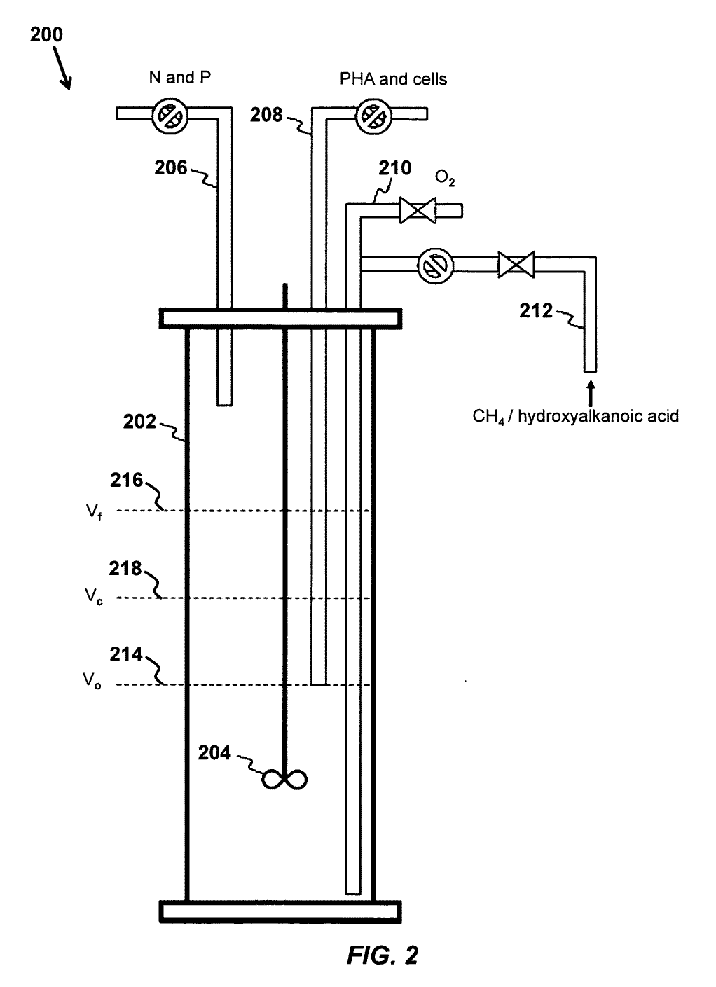

FIG. 2 shows another embodiment of a sequencing batch reactor 200 for PHB production from methane and hydroxyalkanoic acid. This exemplary design provides pH, DO (mixing), and temperature control. The reactor includes a vessel 202, a mixer 204, a valved nutrient inlet 206, a valved PHB and waste outlet 208, an oxygen inlet 210, and a valved methane/hydroxyalkanoic acid inlet 212.

According to one method of PHB production, during a first period, nutrients (e.g., N and P) are added through opened inlet 206 while methane/hydroxyalkanoic acid inlet 212 and harvesting outlet 208 are closed. The mixture volume increases during this period, causing the mixture level in the reactor to rise from the base level V₀ 214. In a second period, methane is added through open inlet 212 and PHB accumulates while nutrient inlet 206 are harvesting outlet 208 are closed. The mixture volume increases further during this period, causing the mixture level in the reactor to rise to the full level V_{f} 216. Although no nutrients are added in the second period, some residual nutrients may still be present in the reactor. In a third period, the cultures are harvested by extracting PHB and waste cells from open harvesting outlet 208 while the nutrient inlet 206 and methane inlet 212 are closed. The volume decreases during this final period, dropping down from level V_{f} 216 to the base level V₀ 214. The cycle then repeats.

According to another method of PHB production, during a first period, nutrients (e.g., N and P) are added through opened inlet 206 while methane/hydroxyalkanoic acid inlet 212 and harvesting outlet 208 are closed. The mixture volume increases during this period, causing the mixture level in the reactor to rise from the base level V₀ 214 to level V_{c} 218. In a second period, nutrients are added through opened inlet 206 and methane/hydroxyalkanoic acid is added through open inlet 212 while harvesting outlet 208 is closed. The mixture volume increases further during this period, causing the mixture level in the reactor to rise from level V_{c} 218 to the full level V_{f} 216. In a third period, methane/hydroxyalkanoic acid is added through open inlet 212 while PHB accumulates in the cells. In a fourth period, the cultures are harvested by extracting PHB and waste cells from open harvesting outlet 208 while the nutrient inlet 206 and methane inlet 212 are closed. The volume decreases during this final period, dropping down from level V_{f} 216 to the base level V₀ 214. The cycle then repeats.

According to another aspect of the invention, cell mass may be extracted from the sequencing reactor, then the extracted portion grown with complete nutrients to increase cell density, and then subjected nutrient limitation. This procedure involves taking samples from the reactor and using the samples for batch incubations to produce PHB.

In one aspect of the invention, bioreactors range from small bench-scale bioreactors to large-scale commercial production bioreactors, and are of various types, including sequencing membrane bioreactors and a continuous multistage dispersed growth configuration. In larger scale bioreactors (i.e., fermentation volumes of tens of liters or more) mass transfer of poorly soluble gases (methane and oxygen) are improved by delivery under pressure or via “dry” fermentations using gas phase delivery of methane and oxygen, and cell densities are increased using ultrafiltration membrane modules (hollow fiber or flat sheet) for cell separation and concentration.

By way of illustration of the principles of the present invention, a specific example of PHB production using a bench-scale bioreactor is described. A bench-scale bioreactor (1-Liter working volume) was cycled daily between periods of 1) methane addition and nitrogen starvation (˜16 hours) and 2) methane starvation with nitrate addition (˜8 hours). A small fraction of the volume (˜50 mL) was sampled twice daily, at the beginning of each period, and was replaced with equivalent media daily. The wasted cells were frozen for analysis of biomass and PHB concentration. The concentration of nitrate in the reactor was monitored daily. Biomass pellets were archived throughout the experiment. DNA was later extracted from these pellets and Terminal Restriction Fragment Length Polymorphism (T-RFLP) with the restriction enzyme Alu I was used to characterize the community within the reactor.

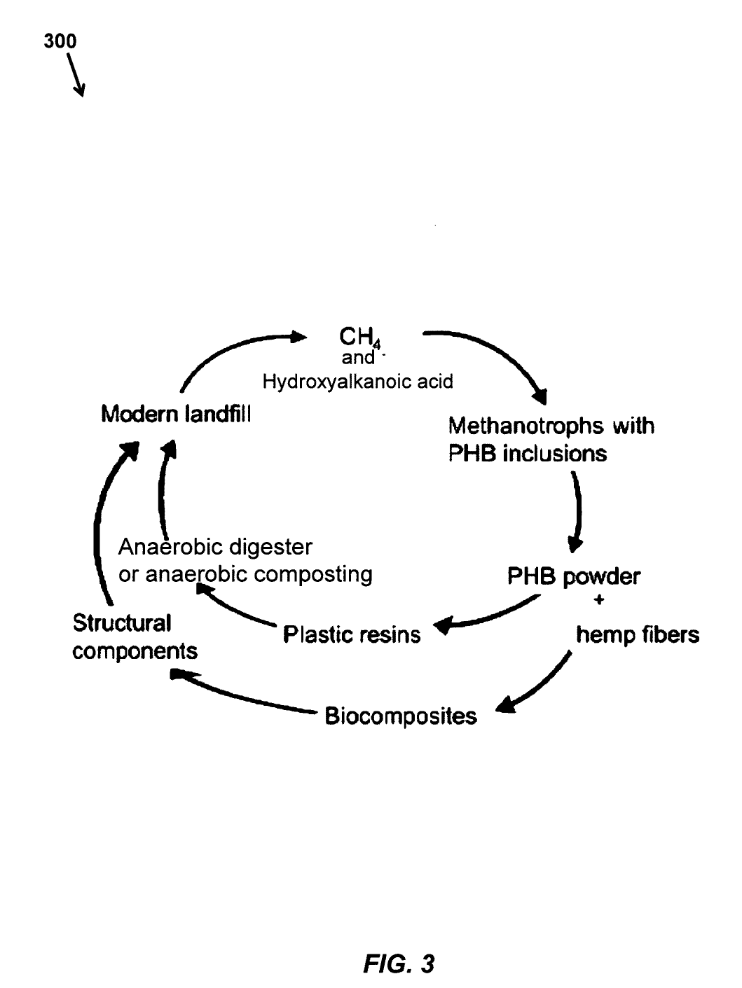

Using the present methods, bioreactors can operate under conditions that select against microorganisms that do not produce PHA, enabling non-sterile production of PHAs and, over the long term, tend to select for organisms that can store PHAs at high levels. The cost of producing PHA using low-cost carbon sources (e.g., products of anaerobic degradation, particularly, methane) and a nonsterile process is expected to be lower than previous production methods. Methane is widely available at low cost, and it is the major product of anaerobic degradation of organic wastes. Moreover, under anaerobic conditions such as those inside a wet landfill or an anaerobic digester, organic wastes including PHB containing products degrade to methane. Aerobic methane-consuming bacteria can convert methane into PHB, completing a “cradle-to-cradle” carbon cycle 300, as shown in FIG. 3. Projected benefits of this cycle include decreased pollution and aesthetic nuisance caused by petrochemical plastics, additional incentives for capture of methane (a major greenhouse gas), decreased CO₂ emissions, decreased energy usage, decreased dependence on petrochemicals, decreased demand for wood, and extended landfill life.

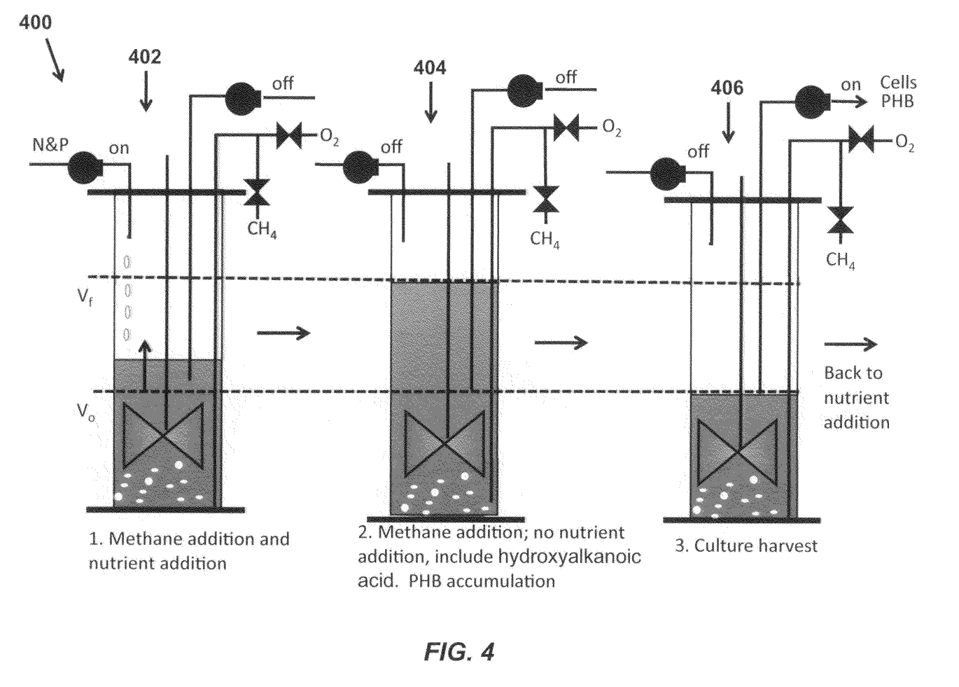

FIG. 4 shows a schematic drawing of PHB production 400 through continuous methane addition with intermittent N addition, where the system provides pH, DO (mixing), and temperature control, according to one embodiment of the current invention. As shown, the method includes methane addition and nutrient addition 402, followed by methane and hydroxyalkanoic acid addition and no nutrient addition 404, resulting in PHB accumulation. Finally, shown is a culture harvest 406, where the cycle returns to nutrient addition.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed:

1. A method of biosynthesis of polyhydroxyalkanoates (PHA), comprising:
a. providing a type II methanotrophic bacteria; and
b. disposing said type II methanotrophic bacteria in an unbalanced growth condition, wherein said unbalanced growth condition comprises a nutrient-deficient media and a hydroxyalkanoic acid, wherein said nutrient-deficient media comprises an absence of an essential nutrient required for cell replication of said type II methanotrophic bacteria.

2. The method of biosynthesis of PHA of claim 1, wherein said type II methanotrophic bacteria comprises pure cultures or mixed cultures of one or more of said type II methanotrophic bacteria.

3. The method of biosynthesis of PHA of claim 1, wherein said essential nutrient is selected from the group consisting of nitrogen, phosphorus, sulfur, iron, sodium, potassium, magnesium, copper, calcium, and manganese.

4. The method of biosynthesis of PHA of claim 1, wherein said hydroxyalkanoic acid is selected from the group consisting of 3-hydroxybutryate (3-HB), 3-hydroxyvalerate (3-HV), and 3-hydroxyhexanoate (3-HHx).

5. The method of biosynthesis of PHA of claim 1, wherein said polyhydroxyalkanoates are selected from the group consisting of 4-hydroxybutryate (4-HB), 4-hydroxyvalerate (4-HV), and 3-hydroxyoctanoate)3-HO).

6. The method of biosynthesis of PHA of claim 1, wherein said hydroxyalkanoic acid is provided with biogas or methane.

7. The method of biosynthesis of PHA of claim 6, wherein said biogas is provided from biodegradation of organic waste.

8. The method of biosynthesis of PHA of claim 1, wherein said hydroxyalkanoic acid is provided with biogas and oxygen, or said biogas and air.

9. The method of biosynthesis of PHA of claim 1, wherein said essential nutrient is provided to said type II methanotrophic bacteria in intermittent pulses.

10. The method of biosynthesis of PHA of claim 1, wherein a bioreactor is used for said biosynthesis of said PHA.

11. The method of biosynthesis of PHA of claim 10, wherein said bioreactor is operated in cycles comprising n and n+1 cycles, wherein each said cycle comprises two periods, wherein in a first period of cycle n, methane is provided in excess to said methanotrophic bacteria in said bioreactor, wherein no nutrients for said methanotrophic bacteria is provided, whereby said methanotrophic bacteria are able to accumulate polyhydroxybutyrate (PHB) and increase in size, wherein in a second period nutrients are provided to said size-increased methanotrophic bacteria, wherein no biogas is provided to said size-increased methanotrophic bacteria, wherein said first period and said second period are repeated for n+1 cycles, whereby repeated cycling through said periods select for bacteria that produce enough said PHB in said first period to replicate during said second period of carbon starvation.

12. The method of biosynthesis of PHA of claim 11, wherein additional species of said methanotrophic bacteria are periodically introduced at a beginning of said first period of said cycle, wherein organisms able to produce more PHBs more quickly become dominant.

13. The method of biosynthesis of PHA of claim 10, wherein said bioreactor is operated in a sterile or non-sterile manner.

14. The method of biosynthesis of PHA of claim 10, wherein a portion of said size-increased methanotrophic bacteria are harvested as waste cells, wherein said PHB is extracted.

15. The method of biosynthesis of PHA of claim 1, wherein a carbon source is supplied continuously to said type II methanotrophic bacteria, wherein said essential nutrient is provided to said type II methanotrophic bacteria in intermittent pulses.

16. The method of biosynthesis of PHA of claim 1 further comprises providing acrylic acid, wherein said acrylic acid is disposed to inhibit beta-oxidation, wherein said acrylic acid comprises prop-2-enoic acid.

17. The method of biosynthesis of PHA of claim 1, wherein said essential nutrient is provided to said type II methanotrophic bacteria in intermittent pulses, wherein either carbon or oxygen are disposed for limiting said growth conditions during said period of nutrient sufficiency, wherein said bacteria is subjected to alternating periods of carbon or oxygen limitation and nutrient limitation.
